(19) United States
(12) Patent Application Publication
(10) Pub. No.: US 2008/0045760 A1
(43) Pub. Date: Feb. 21, 2008

(54) METHOD AND APPARATUS FOR STEAM DEALKYLATION OF HYDROCARBONS IN AN OLEFIN PLANT

(75) Inventors: Helmut Fritz, Muenchen (DE); Volker Goeke, Wolfratshausen (DE)

Correspondence Address: CROWELL & MORING LLP;INTELLECTUAL PROPERTY GROUP, P.O. BOX 14300, WASHINGTON, DC, 20044-4300, US

(73) Assignee: Linde Aktiengesellschaft, Wiesbaden (DE)

(21) Appl. No.: 11/840,896

(22) Filed: Aug. 17, 2007

(30) Foreign Application Priority Data
Aug. 18, 2006 (DE) 10 2006 038 893.3
Dec. 12, 2006 (DE) 10 2006 058 528.3

(51) Int. Cl. C07C 13/20

(52) U.S. Cl. 585/15; 585/402; 585/410; 585/487

(57) ABSTRACT

A method and apparatus for treating a fraction consisting predominantly of hydrocarbons having at least seven carbon atoms (C₇₊ fraction) as produced in a plant for generating hydrocarbons from the steam reforming of hydrocarbon-containing starting material (olefin plant), is disclosed. The C₇₊ fraction is conducted to steam dealkylation following hydration where the useable products benzene and hydrogen are produced.

This application claims the priority of German Patent Documents No. 10 2006 038 893.3, filed Aug. 18, 2006, and No. 10 2006 058 528.3, filed Dec. 12, 2006, the disclosures of which are expressly incorporated by reference herein.

### BACKGROUND AND SUMMARY OF THE INVENTION

The invention relates to a method for treating a fraction consisting predominantly of hydrocarbons having at least seven carbon atoms (C₇₊ fraction), as produced in a plant for generating hydrocarbons from steam reforming of hydrocarbon-containing starting material (olefin plant) and an apparatus for carrying out the method.

In an olefin plant for the steam reforming of hydrocarbon-containing starting (feedstock) material, the hydrocarbon-containing starting material is mixed with steam and for a short time heated to very high temperatures (approx. 850° C.), by which the longer chain hydrocarbons in the starting material are reformed into shorter chain hydrocarbons. These shorter chain hydrocarbons (predominantly ethane) are the primary product of a plant of this type. In addition, a series of by-products is created whose relative percentage and composition depend on the composition of the hydrocarbon-containing starting material.

One of the primary by-products is pyrolysis gasoline. It is highly aromatic (30% benzene, 15% toluene, 20% C8 aromatics), contains many olefins and conjugated diolefins and is separated in the plant from the residual product stream as a fraction which consists predominantly of hydrocarbons having at least five carbon atoms (C₅₊ fraction). As the economically usable component, the C₅₊ fraction contains aromatics which can be used as starting materials for the synthesis of numerous plastic materials and to increase the knock resistance of gasoline. In accordance with the state of the art, the C₅₊ fraction initially undergoes selective hydration, in which the diolefins and styrenes are converted into their respective olefins or ethyl benzenes. Then a separation by distillation of the C₅₊ fraction takes place into a fraction containing predominantly hydrocarbons having five carbon atoms and a fraction which contains predominantly at least six carbon atoms (C₆₊ fraction). The resulting C₆₊ fraction undergoes hydration to convert and remove components containing sulfur, nitrogen and/or oxygen. The now hydrated C₆₊ fraction is, in accordance with the prior art, separated by distillation into a fraction which contains predominantly hydrocarbons having six carbon atoms and a fraction which contains predominantly hydrocarbon having at least seven carbon atoms (C₇₊ fraction). From the fraction which contains predominantly hydrocarbons having six carbon atoms, economically useful benzene can be extracted by means of extractive rectification. To increase the benzene yield, in accordance with the prior art, the C₇₊ fraction undergoes hydro-dealkylation.

A method of this kind for hydro-dealkylation is described, for example, in WO20050071045. The C₇₊ fraction is contacted with hydrogen in the presence of a catalyst at a temperature of 400° C. to 650° C. and at a pressure between 20 bar and 40 bar, with the hydrogen being present in a molar excess of three to six times the hydrocarbons. Under these conditions, the alkyl groups of the individual alkylated aromatics (such as toluene and xylene) are split off so that benzene and the specific alkenes (such as methane and ethane) form.

The consumption of hydrogen in the hydro-dealklylation of the C₇₊ fraction and the costly extractive rectification of the fraction which contains predominantly hydrocarbons having six carbon atoms has a negative effect on the economics of this method from the prior art for extracting benzene.

### BRIEF DESCRIPTION OF THE DRAWING

FIG. 1 illustrates an embodiment of an apparatus in accordance with the principles of the present invention.

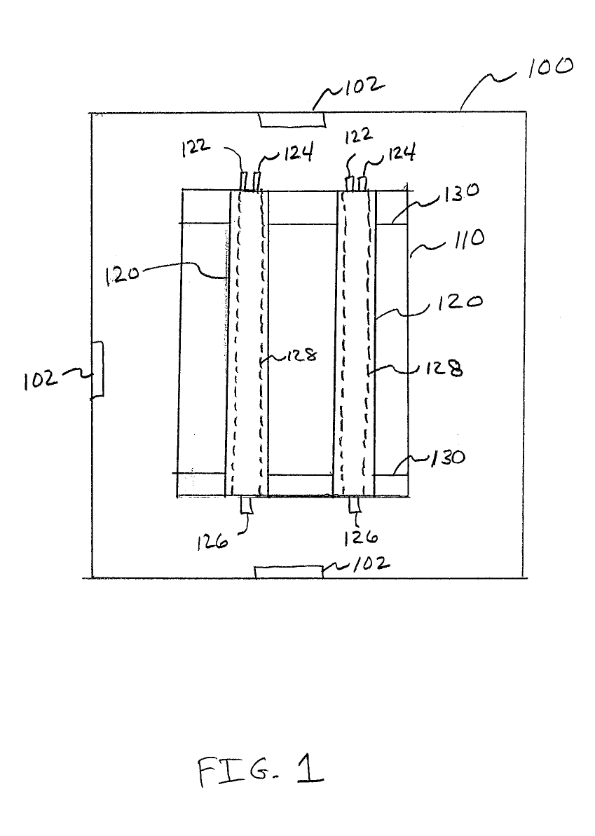

### DETAILED DESCRIPTION OF THE EXEMPLARY EMBODIMENTS

In accordance with the invention, with respect to the method, the C₇₊ fraction undergoes steam dealkylation in which primarily the two usable product materials benzene and hydrogen are produced in addition to by-products such as carbon monoxide and carbon dioxide.

The fundamental concept of the invention is to perform the dealkylation of the alkylated aromatics while generating benzene with the aid of steam dealkylation. Steam dealkylation requires only inexpensive steam as the starting material and produces the valuable by-product hydrogen in addition to the desired quality product benzene.

The C₇₊ fraction used in the steam dealkylation contains mainly:
a) aromatic hydrocarbons having seven to ten carbon atoms,
b) cyclic paraffins (cycloalkenes) having six to ten carbon atoms,
c) iso- and n-paraffins having six to ten carbon atoms,
d) alkenes having seven to ten carbon atoms, or
   any mixture of the preceding, in which the exact composition of the mixture depends on the composition of the specific hydrocarbon-containing starting material from the olefin plant. A starting material consisting more of shorter-chain hydrocarbons in the steam reforming of the olefin plant has a clearly smaller percentage of aromatics in the separation gas than a starting material containing more longer-chain hydrocarbons. The method in accordance with the invention is suitable for each of the compounds of the C₇₊ fractions described.

The hydrocarbons from the C₇₊ fraction advantageously react with steam in the gas phase with the introduction of heat on a solid catalyst. The gaseous C₇₊ fraction is dealkylated by the presence of gaseous water (steam) on a catalyst with the constant introduction of heat, whereby the desired products benzene and hydrogen are produced in addition to carbon monoxide, carbon dioxide and additional by-products.

Preferably the heat required for the dealkylation reaction is generated from combustion of a starting material with air. It proves to be particularly advantageous to use gaseous reaction by-products from the steam dealkylation, specifically carbon monoxide and methane as the starting material for combustion with air. One part of the gaseous reaction by-products from the steam dealkylation, specifically carbon monoxide and methane, is combustible and can thus serve as starting material for combustion to generate the required reaction heat. This saves heating gas and this otherwise unused part of the reaction products can be employed in a more meaningful way.

Following compression in pressure swing adsorption, the gaseous reaction products are expediently separated into gaseous hydrogen and gaseous reaction by-products, specifically carbon monoxide, carbon dioxide and methane. The valuable by-product hydrogen is also present in gaseous form and can be employed much more usefully than in combustion. Through an adsorptive alternating pressure process following compression, the hydrogen can easily be separated from the combustible gaseous reaction by-products which can serve as starting material in the combustion.

Advantageously the flue gases generated in the combustion are cooled through a heat exchanger while heating the starting materials for the steam dealkylation. By using the heat from the flue gases to preheat the starting materials (C₇₊ fraction and steam) for the steam dealkylation, the necessary heat which has to be brought in to maintain the temperatures required for the dealkylation reaction is reduced. This achieves an economical use of energy resources.

The C₇₊ fraction and the steam are advantageously taken past the solid catalyst in pipes, preferably from top to bottom, with the catalyst being located inside the pipes. Heat is expediently brought to the pipes from the outside. The heat required for the dealkylation reaction is advantageously transferred to the pipe by electromagnetic radiation, thermal radiation and/or convection. The actual dealkylation reaction takes place inside the pipe where the catalyst is located. The two components in the reaction (C₇₊ fraction and steam) are taken from top to bottom through the pipes filled with the catalyst. The heat required for the dealkylation reaction is generated outside the pipes and transferred to the pipe by the mechanisms named from which the heat is transferred by means of conduction and convection into the interior of the pipes where the reaction is taking place.

Preferably a solid catalyst of a porous carrier material is used, in particular γ-Al₂O₃, MgAl spinel and/or Cr₂O₃, and an active component on the surface of the carrier material, in particular Rh with 0.1-1.0% loading by weight and/or Pd with 0.2-2.0% loading by weight.

The steam dealkylation is advantageously performed at a temperature of 400° C. to 600° C., preferably 450° C. to 550° C., particularly preferably 480° C. to 520° C. and at a pressure of 1 to 15 bar, preferably 1.2 to 10 bar, particularly preferably 1.5 to 8 bar.

The steam dealkylation is expediently performed at a molar quotient of steam to hydrocarbons which lies in the range from 1 to 20, preferably from 2 to 15, when it enters the reactor. In another embodiment of the invention, the steam dealkylation is performed at a molar quotient of steam to hydrocarbons which lies in the range from 3 to 12, preferably from 5 to 10, when it enters the reactor. Generally the steam dealkylation is performed with a molar excess of water, where the exact ratio in the different embodiments of the inventions depends on the precise composition of the C₇₊ fraction.

It proves advantageous to subject the C₇₊ fraction before steam dealkylation to a process to convert dienes and styrenes, where specifically hydrating methods consuming hydrogen are employed. In another embodiment of the invention, the C₇₊ fraction is separated before steam dealkylation from a fraction of hydrocarbons having at least six carbon atoms where the fraction of hydrocarbons having at least six carbon atoms is subjected to a process to convert dienes and styrenes, specifically a hydrating process which consumes hydrogen. By employing the hydrating methods, any diolefins present in the C₇₊ fraction are converted into their corresponding olefins, just as components containing sulfur, nitrogen and oxygen can be converted and removed. Deactivation of the catalyst is reduced and the life of the catalyst is clearly increased. Depending on the embodiment of the invention, the C₇₊ fraction itself can be hydrated or be separated from a hydrated C₆₊ fraction.

The reaction products from the steam dealkylation are preferably cooled and separated in a 3-phase separation into gaseous reaction products, hydrocarbons and water. The reaction products coming from the steam dealkylation contain not only the desired quality products benzene and hydrogen but also reaction products such as carbon monoxide and carbon dioxide and reaction by-products. To obtain the desired quality products, the reaction products must be separated. This is done by way of a 3-phase separation of the cooled reaction products into gaseous reaction products, in particular hydrogen, carbon monoxide, carbon dioxide and methane, into hydrocarbons, in particular benzene, and into water.

The hydrogen generated in the steam dealkylation of the C₇₊ fraction is expediently fed completely or partially into the starting material for the hydrogen-consuming processes. The hydrogen generated in the steam dealkylation can be used entirely or partially for the hydrogen-consuming processes described in the previous section so that the need for hydrogen to be supplied externally is minimized.

In another embodiment of the invention, the hydrogen generated in the steam dealkylation of the C₇₊ fraction is taken as the starting material for any number of other hydrogen-consuming hydration processes for products and by-products from the olefin plant, in particular to saturate fractions consisting predominantly of hydrocarbons having four or more carbon atoms. The hydration of the C₇₊ fraction is not the only hydrogen-consuming process in an olefin plant. Hydration processes are necessary for the primary products of the olefin plant for which the hydrogen generated in steam dealkylation can likewise be used.

In a further embodiment of the invention, the hydrogen generated in the steam dealkylation of the C₇₊ fraction is taken to an oil refinery as starting material.

Reduction of the sulfur content in the C₇₊ fraction to below 10 ppm, preferably to below 3 ppm, particularly preferably to below 1 ppm, before steam dealkylation proves advantageous for a good yield of the desired reaction product benzene from steam dealkylation.

Preferably the benzene is separated from the hydrocarbons of the reaction products through rectification. Following rectification, the benzene advantageously undergoes adsorptive fine cleaning to dry and remove the trace components, where the benzene is directed across an adsorbent on which the trace components, as opposed to benzene, are adsorbed. By applying the inventive method, the benzene can be extracted from the reaction products by simple rectification and processed further or marketed. Expensive extraction or extractive rectification as when applying a process in accordance with the prior art is not necessary, thus reducing investment and process costs.

Advantageously components boiling close to benzene or components forming azeotropes in the C₇₊ fraction are converted by the steam dealkylation. All reaction products boiling heavier than benzene from rectification, consisting predominantly of non-converted feedstock from the steam deakylation are expediently returned to steam dealkylation through optional hydration as feedstock. In another embodiment of the invention, all reaction products boiling heavier than benzene from rectification, consisting predominantly of non-converted feedstock from steam dealkylation are returned for hydration of the C₇₊ fraction, the C₆₊ fraction or to hydration of a fraction consisting predominantly of hydrocarbons having at least five carbon atoms prior to steam dealkylation. By returning the non-converted feedstocks for hydration or for steam dealkylation, circulation is achieved without losing valuable feedstocks.

In another embodiment of the invention, prior to steam dealkylation a fraction of hydrocarbons having at least eight carbon atoms is separated by distillation from the C₇₊ fraction, where the separated fraction of hydrocarbons having at least eight carbon atoms undergoes separate steam dealkylation. In this embodiment of the invention, xylene (contained predominantly in the separated fraction of hydrocarbons having at least eight carbon atoms) and toluene (contained predominantly in the remaining C₇₊ fraction) undergo separate steam dealkylation.

Concerning the apparatus, the object of the invention is achieved by the apparatus comprising an oven 100 with a furnace 110 and pipes 120 located in the furnace. The actual steam dealkylation takes place in the pipes which in turn are located in the furnace of the oven where the heat required for steam dealkylation can be generated.

The pipes are advantageously installed vertically in the furnace and have heat expansion compensating elements 130 at the lower and/or upper end. The heat expansion compensating elements at the lower and/or upper end of the vertical pipes prevent mechanical stress from temperature differences which can lead to increased wear of the pipes.

Each pipe expediently has a supply for the C₇₊ fraction and the steam, 122, 124, respectively, and an outlet 126 for the reaction products.

It similarly proves advantageous that each pipe is filled on the inside with a catalyst 128, where the catalyst consists of a porous carrier material, in particular γ-Al₂O₃, MgAl spinel and/or Cr₂O₃ and an active component on the surface of the carrier material, in particular Rh with 0.1-1.0% loading by weight and/or Pd with 0.2.-2.0% loading by weight.

Preferably the oven has at least one burner 102 on the wall, the ceiling and/or the floor. The pipes are expediently suitable for an internal pressure of 1 to 15 bar, preferably 1.2 to 10 bar, particularly preferably 1.5 to 8 bar, and for use in an oven with flame temperatures of up to 1400° C.

The present invention is successful specifically in creating an economical alternative to the prior art for treating a C₇₊ fraction. Through the application of the inventive method and the inventive apparatus, the valuable by-product hydrogen is generated in addition to the usable product benzene.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for treating a fraction consisting predominantly of hydrocarbons having at least seven carbon atoms (C₇₊ fraction) as produced in a plant for generating hydrocarbons from steam reforming of hydrocarbon-containing feedstock, wherein the C₇₊ fraction undergoes steam dealkylation, wherein two usable product materials benzene and hydrogen are produced in addition to reaction products such as carbon monoxide and carbon dioxide.

2. The method according to claim 1, wherein the C₇₊ fraction contains:
a) aromatic hydrocarbons having seven to ten carbon atoms;
b) cyclic paraffins (cycloalkenes) having six to ten carbon atoms;
c) iso- and n-paraffins having six to ten carbon atoms;
d) alkenes having seven to ten carbon atoms; or
any mixture of the aforementioned.

3. The method according to claim 1, wherein the hydrocarbons from the C₇₊ fraction react with water in a gas phase when heat is introduced to a solid catalyst.

4. The method according to claim 1, wherein heat required for the dealkylation reaction is generated by combustion of a starting material with air.

5. The method according to claim 1, wherein gaseous reaction products from the steam dealkylation following compression are separated by way of pressure swing adsorption into gaseous hydrogen and gaseous reaction by-products, in particular carbon monoxide, carbon dioxide and methane.

6. The method according to claim 5, wherein the gaseous reaction by-products from the steam dealkylation, in particular carbon monoxide and methane, are also used as starting material for combustion with air.

7. The method according to claim 1, wherein flue gases created during combustion are cooled by a heat exchanger while heating starting materials for the steam dealkylation.

8. The method according to claim 1, wherein the C₇₊ fraction and the steam are directed past a solid-bed catalyst in pipes where the catalyst is on an inside of the pipes.

9. The method according to claim 8, wherein heat is brought to the pipes from outside.

10. The method according to claim 9, wherein the heat required for steam dealkylation is transferred by electromagnetic radiation, thermal radiation and/or convection.

11. The method according to claim 1, wherein a solid-bed catalyst of a porous carrier material is used, in particular γ-Al₂O₃, MgAl spinel and/or Cr₂O₃ and an active component on a surface of the carrier material in particular Rh with 0.1-1.0% loading by weight, and/or Pd with 0.2.-2.0% loading by weight.

12. The method according to claim 1, wherein the steam dealkylation is carried out at a temperature of 400° C. to 600° C., preferably 450° C. to 550° C., particularly preferably 480° C. to 520° C.

13. The method according to claim 1, wherein the steam dealkylation is carried out at a pressure of 1 to 15 bar, preferably 1.2 to 10 bar, particularly preferably 1.5 to 8 bar.

14. The method according to claim 1, wherein the steam dealkylation is carried out at a molar quotient of steam to hydrocarbons which is in a range from 1 to 20, preferably from 2 to 15, when it enters a reactor.

15. The method according to claim 1, wherein the steam dealkylation is carried out at a molar quotient of steam to hydrocarbons which is in a range from 3 to 12, preferably from 5 to 10, when it enters a reactor.

16. The method according to claim 1, wherein the C₇₊ fraction prior to the steam dealkylation undergoes a process to convert dienes and styrenes, where in particular hydrating processes which consume hydrogen are used therefor.

17. The method according to claim 1, wherein the C₇₊ fraction is separated prior to steam dealkylation from a fraction of hydrocarbons having at least six carbon atoms (C₆₊ fraction), where the C₆₊ fraction undergoes a process to convert dienes and styrenes, where in particular hydrating processes which consume hydrogen are used therefor.

18. The method according to claim 1, wherein the C₇₊ fraction undergoes a process prior to the steam dealkylation to convert and remove components containing sulfur, nitrogen and/or oxygen, where in particular hydrating processes which consume hydrogen are used therefor.

19. The method according to claim 1, wherein the reaction products from the steam dealkylation are cooled and separated in a 3-phase separation into gaseous reaction products, hydrocarbons and water.

20. The method according to claim 16, wherein the hydrogen produced in the steam dealkylation of the C₇₊ fraction is fed partially or completely into a starting material for the processes which consume hydrogen.

21. The method according to claim 17, wherein the hydrogen produced in the steam dealkylation of the C₇₊ fraction is fed partially or completely into a starting material for the processes which consume hydrogen.

22. The method according to claim 1, wherein the hydrogen produced in the steam dealkylation of the C₇₊ fraction is fed as starting material to a hydration process of products and by-products from the plant that consumes hydrogen, in particular to a process to saturate fractions consisting predominantly of hydrocarbons having four or more carbon atoms.

23. The method according to claim 1, wherein the hydrogen produced during the steam dealkylation of the C₇₊ fraction is taken to a petroleum refinery as starting material.

24. The method according to claim 1, wherein a sulfur content in the C₇₊ fraction is reduced to below 10 ppm, preferably below 3 ppm, particularly preferably below 1 ppm prior to the steam dealkylation.

25. The method according to claim 1, wherein the benzene is separated from the hydrocarbons by way of rectification of the reaction products.

26. The method according to claim 25, wherein the benzene undergoes absorptive fine cleaning following rectification to dry and remove trace components, where the benzene is directed across an absorbent on which the trace components are adsorbed.

27. The method according to claim 1, wherein components in the C₇₊ fraction boiling close to benzene or forming azeotropes are converted by steam dealkylation.

28. The method according to claim 25, wherein all reaction products from the rectification which are heavier boiling than benzene consisting predominantly of non-converted starting materials from the steam dealkylation are returned by way of an optional hydration to the steam dealkylation as starting material.

29. The method according to claim 25, wherein all reaction products from the rectification which are heavier boiling than benzene consisting predominantly of non-converted starting materials from the steam dealkylation are returned to hydration of the C₇₊ fraction, a C₆₊ fraction or to hydration of a fraction consisting predominantly of hydrocarbons having at least five carbon atoms prior to steam dealkylation.

30. The method according to claim 1, wherein prior to the steam dealkylation a fraction of hydrocarbons having at least eight carbon atoms is separated from the C₇₊ fraction by distillation, where the separated fraction of hydrocarbons having at least eight carbon atoms undergoes separate steam dealkylation.

31. An apparatus for treating a fraction consisting predominantly of hydrocarbons having at least six carbon atoms (C₇₊ fraction) as produced in a plant for generating hydrocarbons from steam reforming of hydrocarbon-containing feedstock, wherein the apparatus includes an oven with a furnace and pipes located in the furnace.

32. The apparatus according to claim 31, wherein the pipes are mounted vertically in the furnace and have heat expansion compensation elements at a bottom and/or a top end.

33. The apparatus according to claim 31, wherein each pipe has a feed for the C₇₊ fraction and the steam and an outlet for reaction products.

34. The apparatus according to claim 31, wherein each pipe is filled on an inside with a catalyst, where the catalyst consists of a porous carrier material, in particular γ-Al₂O₃, MgAl spinel and/or Cr₂O₃ and an active component on a surface of the carrier material in particular Rh with 0.1-1.0% loading by weight, and/or Pd with 0.2.-2.0% loading by weight.

35. The apparatus according to claim 31, wherein the oven has at least one burner on a wall, a ceiling and/or a floor.

36. The apparatus according to claim 31, wherein the pipes are suitable for an internal pressure of from 1 to 5 bar, preferably 1.2 to 10 bar, particularly preferably 1.5 to 8 bar, and for use in an oven with flame temperatures of up to 1400° C.

37. A method of extracting benzene from a hydrocarbon having at least seven carbon atoms, comprising the steps of:
subjecting the hydrocarbon having at least seven carbon atoms to steam dealkylation; and
producing benzene from the steam dealkylation.

38. The method according to claim 37, further comprising the step of producing hydrogen from the steam dealkylation.
